(19) United States
(12) Patent Application Publication
(10) Pub. No.: US 2025/0161263 A1
(43) Pub. Date: May 22, 2025

(54) USE OF A COMPOUND, PHARMACEUTICAL FORMULATION, METHOD OF TREATMENT FOR ASTHMA AND/OR COPD, AND COMPOUND

(71) Applicant: FUNDAÇÃO OSWALDO CRUZ, RIO DE JANEIRO (BR)

(72) Inventors: Milena Botelho Pereira SOARES, Salvador (BR); Renan Fernandes do Espirito SANTO, Salvador (BR); Cristiane Flora VILLARREAL, Salvador (BR); Paulo Vitor França LEMOS, Salvador (BR); Rafael dos Santos COSTA, Salvador (BR)

(21) Appl. No.: 18/836,600

(22) PCT Filed: Feb. 11, 2022
(86) PCT No.: PCT/BR2022/050047

§ 371 Date: Aug. 7, 2024

(51) Int. Cl. A61K 31/37; A61P 11/00; A61P 11/06

(52) U.S. Cl. CPC A61K 31/37; A61P 11/00; A61P 11/06

(57) ABSTRACT

The present invention is part of the field of pharmacology and medicine and relates to the use of coumarin derivative compounds for the treatment of asthma and/or chronic obstructive pulmonary disease (COPD). The present inventors identified that inhaled braylin has high therapeutic efficacy in the treatment of asthma and COPD, with efficacy comparable to systemic dexamethasone, and presenting itself as an unprecedented alternative to conventional treatments.

### FIELD OF INVENTION

The present invention is part of the field of pharmacology and medicine and refers to the use of coumarin-derived compounds for the treatment of asthma and/or chronic obstructive pulmonary disease (COPD).

### BACKGROUND OF INVENTION

The most common diseases in society are those that are directly, or indirectly, linked to the respiratory system. The manifestation of these illnesses is very broad and has different causes, which may be associated with genetic factors, pollution and bad habits (smoking). Of these respiratory diseases, asthma and chronic obstructive pulmonary disease (COPD) are the most common in the world population (WHO, 2021).

Due to this high occurrence, medicine is advancing with new methods of treatment for respiratory diseases. Thus, in order to improve the effectiveness of therapy, new drug formulations are presented to the population, with natural and synthetic compositions.

Even with biotechnological progress in treating respiratory diseases, there are still barriers to be overcome. In this sense, it is common in drug-based therapies to present varied adverse effects, from weight gain to heart problems.

In the early 90s, asthma was defined as a syndrome characterized by variable and reversible airway obstructions, accompanied by an abnormal increase in the responsiveness to various stimuli. However, despite the existence of a definition, there was still no consensual definition for the disease, due to the different causes, mechanisms and responses to therapies (BOUSQUET; CHANEZ; LACOSTE; BARNEON et al., 1990). Currently, according to the National Heart, Lung and Blood Institute (NHLBI), asthma is a chronic lung disease that inflames and constricts the airways. It is common to observe recurrent periods of wheezing, chest tightness, shortness of breath and coughing in asthmatic patients (NIH, 2007). The guidelines for asthma management from the Brazilian Society of Pulmonology and Tisiology add in their definition that in addition to chronicity and tissue inflammation in asthma, there is the participation of many cells and cellular elements (SBPT, 2012).

Asthma, among other chronic respiratory diseases, is an important public health problem having a major negative impact on the population (BOUSQUET; BOUSQUET; GODARD; DAURES, 2005). The numbers regarding the prevalence of asthma in the world are impressive and according to the Global Asthma Network (2014) it affects 334 million people, being the 14th most important disease in the world in terms of extent and duration of disability caused, in addition to being the most difficult to control in children and the elderly.

It is also known that spending on the use of health services and medications by asthmatic patients is twice as high among patients with uncontrolled asthma than among those with controlled asthma, with the lack of asthma control being the factor with the greatest influence on the use of health services, increasing spending proportionally to the severity of the disease (SBPT, 2012). Regarding the impact of asthma on family income, spending currently reaches 25% in patients from lower-income classes, which is 20% higher than the recommendation of the World Health Organization, which is that these expenses should not exceed 20%.

Chronic Obstructive Pulmonary Disease (COPD) is a chronic inflanmmatory lung disease that causes obstruction of the air passages in the lungs. COPD is usually caused by prolonged exposure to particulate matter or irritating gases, often cigarette smoke. Emphysema and chronic bronchitis are the two clinical manifestations that most contribute to COPD and usually occur at the same time. People with COPD have an increased risk of developing heart disease, lung cancer, and a variety of conditions. (MAYO CLINIC, 2020).

Although asthma and COPD are different diseases, they have important pathophysiological similarities, including sharing pharmacotherapeutic strategies. Both are chronic inflammatory diseases of the airways and cause airflow limitation, being characterized by excess mucus production, airway hypersensitivity and bronchoconstriction (JEFFERY, 2000; BUIST, 2003; Widdicombe 2003). The increased production of mucus due to goblet cell hyperplasia in the airways and mucus hypersecretions result in the process of airway narrowing in both diseases (ATHANAZIO, 2012). Immunohistopathological features shared between asthma and COPD include activation and infiltration of common inflammatoiy cells and the dysregulation of inflammatory mediators. For example, eosinophils, which are central effector cells in the development of asthma, are involved in the pathophysiology of COPD, actively participating in the process and being determinants of COPD exacerbations. Around 25-40% of COPD patients have the eosinophilic endotype (LI et al., 2021).

Asthma and COPD are closely associated with the Th2 type immune response, involving eosinophilia, mastocytosis and elevation of IgE levels triggered by allergens (SILVEIRA; NUNES; CARA; SOUZA et al., 2002). When the allergen comes into contact with antigen-presenting cells, it is captured and processed, allowing its presentation to CD4+T lymphocytes via MHCII, leading to their activation and differentiation into Th2 lymphocytes (SILVA & VARGAFTIG, 2005).

Although inflammation is a relevant aspect of asthma and COPD, the chronic inflammatory process characteristic of these diseases is quite complex and differentiated. Unlike what is observed in acute inflammatory responses, all cells of the respiratory system participate in the typical changes of asthma, including constituent cells, such as epithelial cells and vascular endothelial cells, which traditionally do not have inflammatory potential.

The acute inflammatory response, such as that induced by tissue damage or chemical agent, involves vascular and cellular responses at the tissue level, in which primary cytokines, such as IL1β, TNFα and IL-6, are produced by the inflammatory cells and involved in the genesis of classic signs of inflammation, such as pain, redness, heat, edema and loss of function.

On the other hand, the lung inflammation seen in asthma involves the activation of inflammatory cells and structural cells of the lung. The products of these cells involved in typical asthma inflammation include Th2 profile cytokines, such as interleukins IL-4, IL-5 and IL-13. All the observed features of lung inflammation and the physiological dysregulation observed in asthma are the final result of the molecular and cellular events involved in sensitization, development of Th2 cells, elaboration of Th2 cytokines and activation of the effector mechanisms of these cytokines, which are responsible for the initiation and maintenance of the pathophysiological processes in asthma.

Based on this pathophysiology, a drug with potential therapeutic action in asthma should be able to inhibit these events and specific pathophysiological pathways, and not just exert anti-inflammatory effects. This statement is largely validated by the fact that non-steroidal anti-inflammatory drugs, the class of anti-inflammatory drugs most used in the world with high efficacy in numerous diseases and inflammatory processes, have no clinical use in the treatment of asthma and COPD.

In this sense, considering that anti-inflammatory and anti-asthmatic properties are distinct, independent and non-predictive, compounds whose anti-inflammatory properties have been demonstrated, for example in models of local inflammation, will probably not be useful in the treatment of asthma and COPD.

When thinking about the treatment of a patient with asthma or COPD, it must be taken into account that the objective is to improve the patient's quality of life, which can be achieved through controlling symptoms and improving or stabilization of lung function. Currently, treatment must necessarily include non-drug measures, associated with pharmacotherapy (MINISTÉRIO DA SAÚDE (Ministry of Health), 2013).

The pharmacological treatment of asthma and COPD has evolved a lot over the last few years, mainly with the inclusion of immunotherapy in the portfolio of options alongside corticosteroids and bronchodilators.

Among the main bronchodilators, we can point out the class of β2 adrenergic agonists that act via selective interaction with β2 receptors present in bronchiolar smooth muscle, causing an increase in intracellular cAMP, which results in relaxation of smooth muscles, increased frequency of ciliary beating and reduction in mucus viscosity (CARDOSO, 2005). Therefore, β2 agonists promote bronchodilation, relieving symptoms resulting from bronchiolar constriction and decreasing resistance to air entry during breathing (FIREMAN, 1995). The use of β2 agonists, however, can lead to tremors, nervous tension, headaches, tachycardia, and muscle cramps (NHS, 2019).

For 40 years, corticosteroids have been used in the treatment of respiratory tract diseases and, today, their dominance is undisputed and the achievements achieved with their use are difficult to challenge scientifically (SUISSA; ERNST; BENAYOUN; BALTZAN et al., 2000). With the ability to reduce bronchial reactivity and restore airway integrity, corticosteroid-based treatment has been the most effective, acting through different mechanisms of action, such as inhibiting the production of cytokines and chemokines, suppressing the production of inflammatory proteins and transcription factors (BARNES, 2001; BOYTON; ALTMANN, 2004).

However, despite their high action when administered systemically, the toxicities associated with the short and long-term use of systemic corticosteroids are extensive and have been well described previously. Toxicities appear to be linked to dose accumulation and duration of therapy. Patients treated with systemic corticosteroids are at increased risk of glucose intolerance, hypertension, development of cataracts, osteoporosis and adrenal insufficiency (SAYIN, 2017; CHEAH, 2020). Furthermore, the degree of respiratory and peripheral muscle weakness has been correlated with the dose and duration of systemic steroid use in chronic treatments. Courses of systemic steroids for exacerbations in COPD have been associated with hyperglycemia, weight gain, insomnia, anxiety and depression (MIRAVITLLES, 2021).

The justification for the use of inhaled corticosteroids is, therefore, multifactorial, as it allows the delivery of a drug directly to the target organ and the ability to use lower cumulative doses of corticosteroid and reduce systemic absorption. Although a complete absence of systemic absorption of inhaled corticosteroids is ideal, this is not the case. Due to first-pass metabolism in the liver, however, virtually none of the commonly used corticosteroids, such as fluticasone propionate and budesonide, are absorbed after passing through the gastrointestinal tract. Therefore, most systemic absorption of inhaled corticosteroids occurs through the lungs. Despite the theoretical benefit of lower systemic corticosteroid levels, there are documented systemic adverse effects of inhaled corticosteroid use, including adrenal suppression, loss of bone mass density, and increased risk of fracture, glaucoma, and skin bruising (HEFFLER, 2018; ALLEN, 2020).

Another disadvantage of treatment with inhaled corticosteroids in asthma and COPD is the fact that their pharmacological effects disappear quickly when treatment is stopped (GUILBERT; MORGAN; ZEIGER; MAUGER et al., 2006). Furthermore, continuous use of corticosteroids can lead to fluid retention, swelling, increased blood pressure, mood problems, gastrointestinal tract problems, and weight gain. The use of inhaled corticosteroids may also increase the occurrence of oral fungal infections and hoarseness.

However, being effective via inhalation is an extremely desired property for new drug candidates intended for the treatment of asthma and COPD.

Inhaled administration of drugs has proven to be a good therapeutic strategy, since it allows achieving a high local concentration of the active ingredient in the lungs, with reduced absorption and, consequently, fewer adverse effects (LIPWORTH, 1996). In fact, the development of inhaled glucocorticoids revolutionized asthma pharmacotherapy from the 50s onwards. And it was in 1956, thanks to the metered dose inhaler, that this method of administration was incorporated into the management of asthmatic patients, and is today considered a key part of the treatment. of this disease (CROMPTON, 2006).

However, only a small fraction of compounds active systemically (such as the intraperitoneal route) are effective via inhalation, as this depends on specific physicochemical properties of the compound. The administration of medication via inhalation is relatively complex. First, the respiratory tract has developed defense mechanisms that aim to keep inhaled materials out of the lungs as well as remove or inactivate them once they have been deposited.

Furthermore, other characteristics of the compound must be observed for adequate inhalation administration. For example, the compound must have an ionization constant (pKa) and Log P which allows it to cross cell membranes and be distributed in lung tissue, but with a limited rate of systemic absorption. The compound must have chemical stability and low binding affinity to P-glycoprotein so that it is not degraded or removed from the tissue into circulation, remaining in the lung tissue long enough to exert its local therapeutic effect (RUGE, 2013; ALI, 2010, EIXARCH, 2010). Therefore, demonstrating the pharmacological properties of a compound administered systemically is not capable of also predicting its effect via inhalation.

Exemplarily, among the compounds that activate glucocorticoid receptors, the majority of them are not active via inhalation. This can be seen by the fact that we have few inhaled glucocorticoids (beclomethasone, fluticasone, budesonide, ciclesonide and mometasone) available for clinical use, while there are dozens of glucocorticoids for systemic use. Having activity via inhalation is a property of the compound, not the class, which depends on the bioavailability of this drug in lung tissue after being administered via this route.

Despite the different strategies for treating and improving the quality of life of asthmatic and COPD patients, the multifactorial nature that can lead to the onset of these diseases makes them increasingly challenging for researchers. As a result, new efforts continue to be made to control it, whether in the discovery of new active molecules, new biological strategies or even new drug delivery systems.

### SUMMARY OF INVENTION

In a first aspect, the present invention relates to the use of a compound of Formula I:

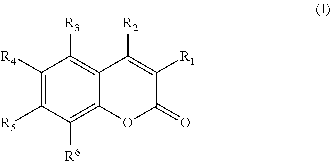

wherein any one of R₁, R₂, R₃, R₄, R₅, R₆, is independently selected from the group consisting of H, OH, O, S, N, C₁-C₅ alkyl, C₂-C₅ alkenyl, C₂-C₅ alkynyl, optionally aromatic C₃-C₇ cycloalkyl, C₁-C₅ heteroalkyl and optionally aromatic C₃-C₇ heterocycloalkyl; wherein any of C₁-C₅ alkyl, C₂-C₅ alkenyl, C₂-C₅ alkynyl, optionally aromatic C₃-C₇ cycloalkyl, C₁-C₅ heteroalkyl and optionally aromatic C₃-C₇ heterocycloalkyl may be optionally substituted with one or more substituents selected from OH, O, S, N, C₁-C₅ alkyl, C₂-C₅ alkenyl, C₂-C₅ alkynyl, optionally aromatic C₃-C₇ cycloalkyl, C₁-C₅ heteroalkyl and optionally aromatic C₃-C₇ heterocycloalkyl; and in which C₁-C₅ heteroalkyl and optionally aromatic C₃-C₇ heterocycloalkyl contain in the chain thereof 1 to 3 heteroatoms selected from F, O, N, Cl, Br, I, S; and/or
R₃ and R₄, R₄ and R₅, R₅ and R₆ are independently taken together to form an optionally aromatic 3 to 7 membered cyclic group which may contain 1 to 3 heteroatoms selected from O, N, S as ring members, the cyclic group being optionally substituted with one or more substituents selected from OH, O, S, N, C₁-C₅ alkyl, C₂-C₅ alkenyl, C₂-C₅ alkynyl, optionally aromatic C₃-C₇ cycloalkyl, C₁-C₅ heteroalkyl and optionally aromatic C₃-C₇ heterocycloalkyl in which C₁-C₅ heteroalkyl and optionally aromatic C₃-C₇ heterocycloalkyl contain in their chain 1 to 3 heteroatoms selected from F, O, N, Cl, Br, I, S;
or salts, prodrugs, stereoisomers, hydrates, dimeric derivatives, isosteres, bioisosteres, and polymorphic forms thereof,
for the manufacture of a medicine for the treatment of an upper airway disease.

In a first embodiment, the compound is selected from the compound of Formula II:

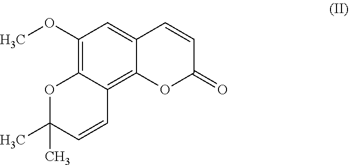 [SMILES: COc1cc2ccc(=O)oc2c2c1OC(C)(C)C=C2]

or salts, prodrugs, stereoisomers, hydrates, dimeric derivatives, isosteres, bioisosteres, and polymorphic forms thereof. In a preferred embodiment, the compound is braylin or its pharmaceutically acceptable salts.

In another embodiment, the medicine is formulated in a form suitable for administration via inhalation.

In another embodiment, the medicine contains from 1 to 1,000 mg of the compound of Formula I.

In yet another embodiment, the medicine is in the form of a powder, fine granules, solution or suspension. In an alternative embodiment, the medicament is formulated in a form suitable for administration by capsule, spray or aerosol.

In a second aspect, the present invention relates to a pharmaceutical formulation comprising a compound of Formula I and at least one pharmaceutically acceptable additive.

In a first embodiment, the formulation comprises the compound of Formula II, or salts, prodrugs, stereoisomers, hydrates, dimeric derivatives, isosteres, bioisosteres, and polymorphic forms thereof. In a preferred embodiment the compound is braylin or its pharmaceutically acceptable salts.

In another embodiment, the formulation is in a form suitable for administration via inhalation.

In yet another embodiment, the pharmaceutical formulation is for the treatment of asthma and/or COPD.

In another embodiment, the pharmaceutical formulation of the invention is in the form of a powder, fine granules, solution or suspension.

In yet another embodiment, the pharmaceutical formulation is in the form of a capsule, spray or aerosol.

In a further embodiment, the pharmaceutical formulation contains from 1 to 1000 mg of the compound of Formula I.

In a third aspect, the invention relates to a method of treating asthma and/or COPD comprising administering a therapeutically effective amount of a compound of Formula I to a patient in need thereof.

In a first embodiment, the compound is selected from the compound of Formula II, or salts, prodrugs, stereoisomers, hydrates, dimeric derivatives, isosteres, bioisosteres, and polymorphic forms thereof. In a preferred embodiment the compound is braylin or its pharmaceutically acceptable salts.

In one embodiment, the compound is administered at a dose of 1 to 100 mg/kg. In a preferred embodiment, the compound is administered at a dose of 50 mg/kg.

In another embodiment, the compound is administered via inhalation.

In another embodiment, the compound is administered in the form of a powder, fine granules, solution or suspension. In an alternative embodiment, the compound is administered by capsule, spray or aerosol.

In another aspect, the present invention relates to a compound of Formula I for the use in the treatment of asthma and/or COPD.

In a first embodiment, the compound comprises the compound of Formula II, or salts, prodrugs, stereoisomers, hydrates, dimeric derivatives, isosteres, bioisosteres, and polymorphic forms thereof. In a preferred embodiment, the compound is braylin or its pharmaceutically acceptable salts.

### BRIEF DESCRIPTION OF THE FIGURES

FIG. 1. Effect of braylin administered by different routes in the airway hypersensitivity model in mice. The X axis represents the groups tested: mice without experimental manipulation (Naive), mice induced to the airway hypersensitivity model treated with vehicle (Ve; 10% propylene glycol in saline), with dexamethasone intraperitoneally (30 mg/kg/ip; gold standard), with braylin 50 mg/kg intraperitoneally (50 ip), and with braylin 50 mg/kg by inhalation (50 in). The Y axis shows the amount of total inflammatory cells (×104) counted in the bronchoalveolar lavage fluid. The treatments were performed for 5 consecutive days, 2 hours before the ovalbumin challenge. The bronchoalveolar lavage fluid was collected for quantifications 24 hours after the last challenge. Cells were quantified in bronchoalveolar lavage fluid under a light microscope using a Neubauer chamber. *Statistically significant difference between the treated groups and the vehicle group (p<0.05). ^{#}Statistically significant difference in relation to the naive group (p<0.05). Data are represented as mean±standard deviation with n=5 animals per group. One-way ANOVA test, followed by Tukey's multiple comparison post-test.

FIG. 2. Dose-response curve of inhaled braylin in the airway hypersensitivity model in mice. The X axis represents the groups tested: mice without experimental manipulation (Naive), mice induced to the airway hypersensitivity model treated with vehicle (Ve; 10% propylene glycol in saline) and braylin (12.5 to 100 mg/kg) by inhalation. Dexamethasone (30 mg/kg) intraperitoneally was the gold standard. The Y axis shows the amount of total inflammatory cells (×104) counted in the bronchoalveolar lavage fluid. The treatments were performed for 5 consecutive days, 2 hours before the challenge with ovalbumin. The bronchoalveolar lavage fluid was collected for quantification 24 hours after the last challenge. The cells were quantified in the bronchoalveolar lavage fluid under a light microscope with the aid of the Neubauer chamber. *Statistically significant difference in relation to the vehicle group (p<0.05). ^{#}Statistically significant difference in relation to the naive group (p<0.05). Data are represented as mean±standard deviation with n=5 animals per group. One-way ANOVA test, followed by Tukey's multiple comparison post-test.

FIG. 3. Effect of braylin on the differential count of inflammatory cells in bronchoalveolar lavage in the airway hypersensitivity model in mice. Panels show representative images of bronchoalveolar lavage cells from (A) naive animals, (B) animals induced in the airway hypersensitivity model and treated with vehicle, (C) animals induced and treated with dexamethasone (30 mg/kg/ip), and (D) animals induced to the model and treated with braylin (50 mg/kg/in). Material stained with hematoxylin and eosin, magnification (100×). Differential quantification of monocytes (E), neutrophils (F) and eosinophils (G) in bronchoalveolar lavage. Data represented as a percentage in relation to the total count. ^{#}Statistically significant difference in relation to the naive group (p<0.05). *Statistically significant difference between the treated groups and the vehicle group (p<0.05). Data represented as mean±standard deviation with n=5 animals per group. One-way ANOVA test, followed by Tukey's multiple comparison post-test.

FIG. 4. Effect of braylin on cytokine levels in bronchoalveolar lavage of mice with airway hypersensitivity. Panels show the levels of cytokines (A) IL-4, (B) IL-5 and (C) IL-13 in the bronchoalveolar lavage of mice, determined by ELISA. The X axis represents the groups tested: mice without experimental manipulation (Naive), mice induced to the airway hypersensitivity model treated with vehicle (Ve; 10% propylene glycol in saline), dexamethasone (30 mg/Kg/ip; gold standard), and braylin (12.5, 50 and 100 mg/kg) via inhalation. Treatments were performed for 5 consecutive days, 2 hours before the ovalbumin challenge. Bronchoalveolar lavage was collected for quantification 24 hours after the last challenge. *Statistically significant difference in relation to the vehicle group (p<0.05). ^{#}Statistically significant difference in relation to the naive group (p<0.05). Data represented as mean±standard deviation with n=6 animals per group. One-way ANOVA test, followed by Tukey's multiple comparison post-test.

FIG. 5. Effects of braylin on lung tissue and cellular parameters. Panels show representative images of mice treated with vehicle (C-D), dexamethasone (E-F; 30 mg/kg/ip) or braylin (G-H; 50 mg/kg/in). Naive animals comprise the naive group (A-B). Lungs stained with HE (A, C, E, G). Lungs stained with periodic acid-Schiff (PAS) (B, D, F, H). Arrowheads indicate cells of the inflammatory infiltrate. Arrows indicate PAS-labeled goblet cells. Magnification 40×, bar 50 μm. Panel I shows the cell counts in the inflammatory infiltrate of the different experimental groups, while panel J shows the quantification of PAS-labeled mucus-producing goblet cells. *Statistically significant difference in relation to the vehicle group (p<0.05). ^{#}Statistically significant difference in relation to the naive group (p<0.05). Data represented as mean±standard deviation with n=5 animals per group. One-way ANOVA test, followed by Tukey's multiple comparison post-test.

### DETAILED DESCRIPTION OF THE INVENTION

Unless defined differently, all technical and scientific terms used herein have the same meaning as understood by a person skilled in the art to which the invention belongs. Conventional techniques of molecular biology and immunology are well known to a person skilled in the art. The specification also provides definitions of terms to assist in the interpretation of what is described herein and the claims. Unless otherwise indicated, all numbers expressing quantities, percentages and proportions, and other numerical values used in the specification and claims, are to be understood as being modified, in all cases, by the term “about”. Thus, unless otherwise indicated, the numerical parameters shown in the specification and in the claims are approximations that may vary depending on the properties to be obtained.

In an attempt to find a new drug that reduces adverse effects in the treatment of asthma and COPD, the present researchers developed characterization work in order to verify the pharmacological potential of a compound from the coumarin group.

The present inventors identified that braylin has high therapeutic efficacy in the treatment of asthma and COPD, comparable to dexamethasone (gold standard drug). The therapeutic effects were dose-dependent. In a model of sensitization and chronic inflammation of the airways, the compounds of the invention, via inhalation, reduced important tissue, biochemical and cellular parameters involved in the pathophysiology of asthma and COPD, namely: they reduced the count of inflammatory cells in bronchoalveolar lavage; reduced the levels of cytokines IL4, IL-5 and IL-13 in bronchoalveolar lavage; reduced the inflammatory infiltrate in lung tissue; reduced mucus production by goblet cells of the bronchiolar epithelium.

The compounds of Formula I according to the present invention can be synthesized or isolated by any methods and chemical routes known and available to a person skilled in the art.

Alternatively, the compounds of Formula I of the present invention are extracted and isolated from roots of Z. tingoassuiba St. Hil according to the method described by Costa and collaborators (COSTA, 2018).

The present invention may also comprise pharmaceutically acceptable salts of the compounds of Formula I. Examples of pharmaceutically acceptable salts that may be formed by the compound of the present invention include inorganic acid salts, such as hydrochloride, hydrobromide, hydroiodide, sulfate, nitrate, phosphate, diphosphate and the like, organic acid salts such as succinate, fumarate, acetate, methanesulfonate, toluenesulfonate and the like, alkali metal salts such as sodium salt, potassium salt and the like, alkaline earth metal salts such as magnesium salt, calcium salt and the like, ammonium salts such as ammonium salt, alkylammonium salt and the like.

In addition, the present application also comprises solvates of compounds of Formula I or their pharmaceutically acceptable salts. Examples of the solvent include water, methanol, ethanol, isopropanol, acetone, ethyl acetate and the like.

Thus, in a first embodiment, the present invention is directed to the use of a compound of Formula I, for the manufacture of a medicine for the treatment of asthma and chronic obstructive pulmonary disease (COPD).

The present compounds, as well as their salts, hydrates and solvates, can also be used as the active ingredient of a pharmaceutical agent of the present invention. The route of administration of the pharmaceutical agent of the present invention is not particularly limited, and the agent can be administered orally, pulmonaryly or parenterally. Preferably, the route of administration of the pharmaceutical agent of the present invention is the inhalation route.

As used herein, the terms “administer,” “administration,” and similar terms, refer to any method that, injudicious medical practice, delivers a compound of interest to an individual in such a manner as to provide a therapeutic effect. A specific aspect of the present invention provides for the inhalational administration of a therapeutically effective amount of the present compounds to a patient in need thereof.

In one embodiment, the compounds of the present invention are administered via inhalation. By “inhalation administration” or “inhalation administration” is meant a mode of administration of the compound that is capable of releasing or delivering the compounds to any part of the individual's respiratory tract. Any part of the respiratory tract means, for example, the mouth, trachea, bronchi, bronchioles, lungs, among others. Preferably, the compound of interest reaches the tracheas, bronchi, bronchioles and/or lungs.

Just like the pharmaceutical agent of the present invention, the compound of the present invention can be directly administered to patients. Preferably, however, it should be administered as a preparation in the form of a pharmaceutical composition containing an active ingredient and at least one pharmaceutically and pharmacologically acceptable additive.

Thus, in a second aspect, the present invention relates to a pharmaceutical formulation comprising at least one compound according to the invention and at least one pharmaceutically acceptable additive.

The composition of interest can be formulated to be compatible with the desired route of administration. The composition may be formulated as a tablet, capsule, solution, powder, inhalant, lotion, tincture, lozenge, suppository, or transdermal patch. Preferably, the composition is formulated as a capsule, solution, powder, inhalant.

As used herein, the pharmaceutically and pharmacologically acceptable additive, for example, may be cited as an excipient, disintegrant or disintegrant auxiliary, binder, coating agent, dye, diluent, base, solubilizer or solubilizer auxiliary, isotonicity agent, pH regulator, stabilizer, propellant, adhesive and the like. Examples of a preparation suitable for parenteral administration include inhalant powder, capsule, powder, fine granule, solution, suspension, aerosol, spray and nebulizer. However, the form of preparation should not be limited to these only.

For administration by the inhalation route, the compounds may be released, for example, in the form of an aerosol spray from a pressurized container dispenser, or not, and may contain a suitable propellant, for example, a gas, or by other known methods. As an example of suitable devices we can mention a metered dose inhaler, pressurized metered dose inhaler, pressurized metered dose inhaler.

Other types of devices may also be suitable for administering the compounds according to the present application. For example, the present compounds can be administered by means of ultrasonic inhalers, dry powder inhalers, soft mist inhalers, nebulizers, capsule inhalers, and any other methods suitable for inhalational administration of the compounds.

A suitable preparation for solid formulations may contain, as an additive, for example, excipients such as glucose, lactose, lactose monohydrate, D-mannitol, starch, cellulose, crystalline cellulose and the like; disintegrant or disintegrant auxiliary, such as carboxymethylcellulose, starch, calcium carboxymethylcellulose, silicon dioxide and the like; binder such as hydroxypropylcellulose, hydroxypropylmethylcellulose, polyvinylpyrrolidone, gelatin and the like; lubricant such as magnesium stearate, talc and the like; base, such as hydroxypropylmethylcellulose, sucrose, polyethylene glycol, gelatin, kaolin, glycerol, purified water, hard fat, and the like.

A suitable preparation for a liquid formulation may contain additives such as a solubilizer or solubilizer auxiliary, capable of constituting an aqueous formulation or a composition to be dissolved when in use, such as in water, distilled water for injection, solution saline, propylene glycol, and the like; isotonicity agent, such as glucose, sodium chloride, D-mannitol, glycerol, and the like; pH regulator such as an inorganic acid, organic acid, inorganic or organic base or the like.

The active agent is preferably administered in an effective amount. As used herein, the phrase “effective amount” refers to the amount of a component which is sufficient to produce a desired therapeutic response without undue adverse side effects (such as toxicity, irritation, or allergic response) commensurate with a reasonable benefit/risk ratio, when used in the manner presently described. For example, a “therapeutically effective amount,” may be a sufficient amount of the active agent to cause regression, control or prevent progression of asthma, chronic obstructive pulmonary disease, and/or symptoms associated with these diseases.

The actual amount administered, and the rate and time course of administration, will depend on the nature and severity of the condition being treated. The prescription of treatment, e.g. decisions on dosage, timing, etc., is within the responsibility of general ones skilled in the art and specialists, and typically considers the disorder being treated, the condition of the individual patient, the site of release, the method of administration and other factors known to those skilled in the art. Examples of techniques and protocols can be found, for example, in Remington's Pharmaceutical Sciences.

Although the dose of the pharmaceutical agent of the present invention should be varied, depending on the type of disease to be applied, patient conditions, such as age, body weight, symptom, and the like, the unit dose is generally about 50-1,000 mg of active ingredient per administration. More specifically, the unit dose may be 150 to 900 mg, 200 to 800 mg, and 400 to 600 mg. In general, the dose mentioned above can be administered in one to several portions per day, or can be administered every few days. In particular, the dosage of the present compounds ranges from 1 to 100 mg/kg. Preferably, the dosage is 50 mg/kg.

Throughout the application, descriptions of various embodiments use the term “comprising,” which will be understood by a person skilled in the art that, in some specific cases, an embodiment may alternatively be described using the language “which consists essentially of” or “which consists.”

Unless otherwise defined all technical and scientific terms used herein have the same meaning as customarily understood by a person skilled in the art to which the present subject matter belongs.

For the purposes of better understanding of the present disclosures and, in no way, limiting the scope of the disclosures, unless otherwise indicated, all numbers expressing quantities, percentages or proportions, and other numerical values used in the specification and claims, are to be understood as being modified in all cases by the term “about”. Consequently, unless otherwise indicated, the numerical parameters presented in the specification and attached claims are approximations that may vary depending on the desired properties sought to be obtained. At a minimum, each numerical parameter must be at least interpreted considering the reported number of significant digits and by applying common rounding techniques.

The present invention is also described by the non-limiting examples below, which are merely illustrative. Various modifications and variations of the embodiments are evident to the person skilled in the art, without departing from the spirit and scope of the invention.

Numerous variations affecting the scope of protection of this application are allowed. In this way, the fact is reinforced that the present invention is not limited to the particular configurations/embodiments described above.

### EXAMPLES

### Example 1

### Materials E Methods

### Extraction, Isolation and Identification of Braylin

Specimens of Z. tingoassuiba St. Hil were collected on Aug. 12, 2009 in the district of Jaiba, municipality of Feira de Santana—Bahia (12° 12′ 52.560″ S; 38° 52′ 46.205″ W). The identification of the specimen was carried out by Professor Maria Lenise da Silva Guedes. With exsicata deposited at the Alexandre Leal Costa Herbarium (ALCB) under number 88005. The extraction, purification and identification procedures for braylin from roots of Z. tingoassuiba St. Hil were described by Costa and collaborators (COSTA, 2018). The formula was established by mass spectroscopy and nuclear magnetic resonance (NMR) analysis to confinn its chemical structure with a purity greater than 99% (YOO, 2002).

### Animals

Male mice of the BALB/c lineage, weighing between 20 and 25 g, from the vivarium of the Goncalo Moniz Research Center, FIOCRUZ/BA. The animals were maintained under controlled temperature conditions (22±2° C.), in a 12-hour light/dark cycle with water and food ad libitun. All protocols and manipulations were approved by the Ethics Committee for animal experimentation at FIOCRUZ (CEUA/FIOCRUZ/L-IGM-015/2013).

### Airway Hypersensitivity Model and Treatments

The ovalbumin-induced airway hypersensitivity model (BOLANDI et al., 2021), used as the basis for the present findings, induces pathophysiological and structural changes which characterize respiratory diseases such as asthma and chronic obstructive pulmonary disease (COPD).

The mice were divided into groups of six animals and immunized with a subcutaneous injection of 10 μg of ovalbumin (Sigma, St. Louis, MO) diluted in 2 mg/ml alum (Alunmlmject; Pierce, Rockford, IL), followed by of a booster injection 14 days later. From day 28 onwards, the mice were placed in an acrylic box and subjected to inhalation exposure to ovalbumin (1%) for 15 minutes per day, for five consecutive days. The ovalbumin solution was nebulized using an ultrasonic inhaler (RespiraMax, Brazil). The protocol used to induce airway hypersensitivity was performed as previously described (POSSA, 2013). The naive group was challenged with saline only. To carry out the treatments, two hours before each challenge via inhalation, the mice were treated with braylin (100, 50, 25.5 and 12.5 mg/kg, via inhalation), dexamethasone (30 mg/kg via intraperitoneal) or vehicle (10% propylene glycol in saline, inhaled).

### Bronchoalveolar Lavage Collection

The animals were euthanized with a lethal dose of ketamine and xylazine (300 mg/Kg and 30 mg/Kg, respectively via i.p.) 24 hours after the last challenge to collect bronchoalveolar lavage (BAL). Intratracheal instillation of 1 ml of ice-cold PBS was performed, followed by BAL collection, and this procedure was repeated. The first wash was centrifuged and the supernatant stored at −70° C. for subsequent quantification of cytokines by ELISA. The second wash was centrifuged, the supernatant was discarded and the pellets were resuspended in 1 ml of saline to count total leukocytes using a Neubauer chamber. To carry out the differential cell count, 10,000 cells were collected from the previous resuspension, centrifuged in Cytospin® and stained with hematoxylin and eosin (VASCONCELOS, 2009).

### Histopathological and Morphometric Analysis

After collecting the bronchoalveolar lavage, lung perfusion was performed intratracheally with 1 ml formalin (4%) and, subsequently, the right lobe of the lungs of each animal was removed and fixed in the same solution for histological and morphometric analysis. The sections were stained with hematoxylin and eosin to quantify inflammatory cells by optical microscopy according to the number of cores present in the fields with the largest population of cells. To quantify mucus production, staining was performed with periodic acid-Schiff (PAS) and the marked areas were quantified. The area of lung tissue labeled with PAS was considered positive for mucus produced by Goblet cells (SOUTHAM, 2008). 10 fields (400×) per animal were analyzed for a total of 5 animals, and the data were used to calculate the average number of cells per mm², or the area stained with PAS. The program used to assist in cell counting and area determination was Image-Pro PLUS, version 4.5 (Media Cybernetics, Silver Spring, USA).

### Quantification of Cytokines in Bronchoalveolar Lavage

The bronchoalveolar lavage supernatant stored at −70° C. was thawed and used to quantify the cytokines IL-4, IL-5 and IL-13 by the ELISA method, using specific kits (R&D System, Minnesota, MN, USA) for mice, following the manufacturer's instructions (VASCONCELOS, 2009).

### Statistical Analysis

The results were expressed as mean±standard deviation. Statistical differences were determined by the one-way ANOVA test, followed by Tukey's post-test, with a significance level previously established at p<0.05. Analyzes were performed using the GraphPad Prism 5.0 program (California, LA, USA).

### Results

### Influence of the Route of Administration on the Bioactivity of Braylin in the Airway Hypersensitivity Model in Mice

With the aim of establishing whether braylin has pharmacological activity when administered via inhalation, the effect of inhalation or intraperitoneal administration of this coumarin on the count of inflammatory cells in bronchoalveolar lavage (BAL) was compared.

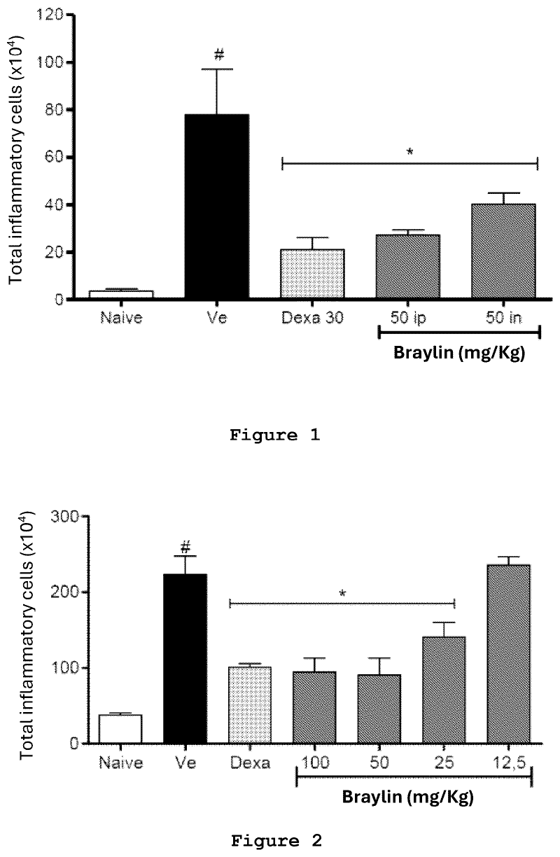

Mice induced in the airway hypersensitivity model by ovalbumin and treated with vehicle showed an increase in the number of total inflammatory cells in the BAL compared to naive animals. The number of inflammatory cells in the BAL was significantly lower (p<0.05) in sick animals treated with braylin (50 mg/kg), both intraperitoneally and by inhalation. A significant inhibition of this parameter was also observed in mice treated with the gold standard drug, dexamethasone at a dose of 30 mg/kg intraperitoneally (FIG. 1).

### Dose Response Curve of Inhaled Braylin in the Airway Hypersensitivity Model in Mice

The dose-dependency relationship of the effect of braylin administered via inhalation in the dose range of 12.5 to 100 mg/kg was then evaluated (FIG. 2). Inhaled braylin at doses of 25, 50 and 100 mg/kg reduced, in a non-dose-dependent manner, the number of inflammatory cells in the BAL of mice with airway hypersensitivity compared to those treated with vehicle (p<0.05). At a dose of 12.5 mg/kg, braylin had no effect. The effect of inhaled braylin had similar efficacy to systemic treatment with dexamethasone (30 mg/kg/ip), considered the gold standard in this trial.

### Braylin Reduces the Presence of Eosinophils and Neutrophils in Bronchoalveolar Lavage

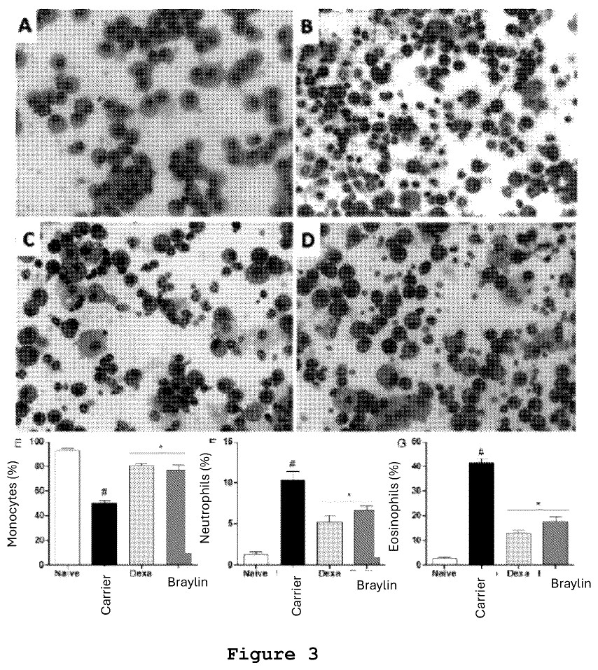

In addition to quantifying the total number of inflammatory cells in the bronchoalveolar lavage, differential quantification between eosinophils, neutrophils and mononuclear cells in these samples was also performed (FIG. 3). Inhalation treatment with braylin (50 mg/Kg) reduced the amount of eosinophils and neutrophils present in BAL compared to lavage from animals treated with vehicle (p<0.05). The quantity of mononuclear cells in animals treated with braylin approached the values found in mice in the naive group, which were not induced to airway hypersensitivity. Dexamethasone (30 mg/kg/ip) induced an effect with a profile similar to that of braylin, with a reduction in the numbers of eosinophils and neutrophils in the BAL.

### Braylin Modulates the Cytokines IL-4, IL-5 and IL-13

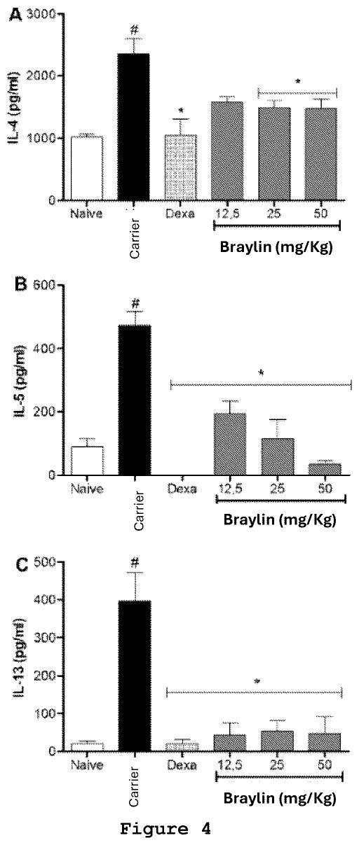

The levels of cytokines that participate in the Th2 response were quantified in the bronchoalveolar lavage of mice from different experimental groups. Mice induced in the airway hypersensitivity model showed elevated levels of IL4, IL-5 and IL-13 cytokines in the BAL compared to naive mice (p<0.05). Inhaled braylin, at doses of 25 and 50 mg/kg, reduced the levels of IL4, IL-5 and IL-13 in BAL (p<0.05). However, braylin at a dose of 12.5 mg/kg induced a significant reduction in IL-5 and IL-13, but not IL-4, in the BAL of mice. Animals treated with systemic dexamethasone (30 mg/kg/ip) showed a reduction in the levels of all cytokines quantified in the BAL (FIG. 4), with a magnitude similar to that obtained with inhaled braylin.

### Braylin Reduces Pulmonary Inflammatory Infiltrate and the Occurrence of Goblet Cell Metaplasia.

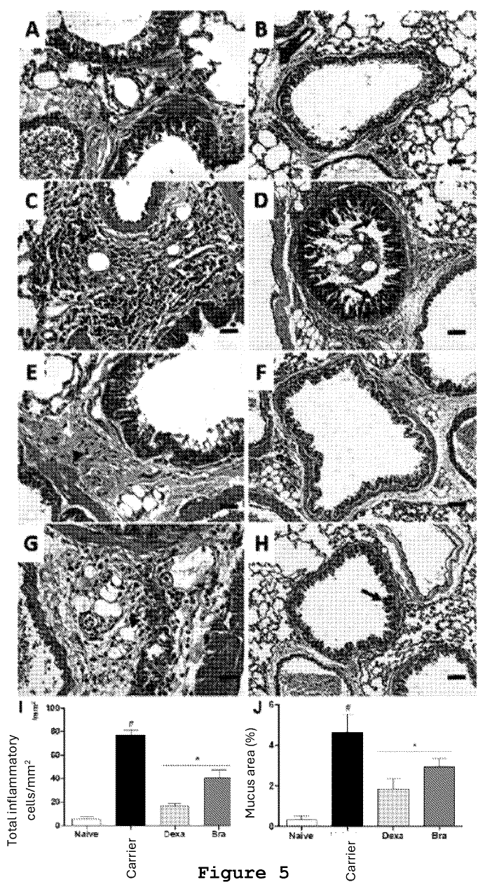

To characterize the tissue changes caused by the induction of the airway hypersensitivity model and the possible effect of braylin on the migration of inflammatory cells, sections of lungs stained with HE were examined. A large cellular infiltrate containing lymphocytes, macrophages and eosinophils was observed in animals induced to the model and treated with vehicle. Mice treated with inhaled braylin at 50 mg/kg had a reduction in lung inflammation with a decrease in the presence of inflammatory cells compared to animals treated with vehicle (p<0.05, FIG. 5I). Systemic treatment with dexamethasone (30 mg/kg/ip) was also able to reduce the pulmonary inflammatory infiltrate. The occurrence of Goblet cell metaplasia in the bronchiolar epithelium was determined by staining the tissue with periodic acid-Schiff (PAS) and shows increased mucus formation. Lungs from mice with airway hypersensitivity treated with vehicle showed a greater area stained with PAS (p<0.05, FIG. 5J). Braylin treatment reduced Goblet cell labeling in the bronchiolar epithelium of animals with induced airway hypersensitivity (p<0.05), indicating its ability to modulate mucus production. As expected, systemic dexamethasone also reduced the presence of mucus in PAS-stained Goblet cells.

### REFERENCES

ABRAMSON, M. J.; WALTERS, J.; WALTERS, E. H. Adverse effects of beta-agonists: are they clinically relevant?Am J Respir Med, 2, no. 4, p. 287-297, 2003.
ALAN, I. S.; ALAN, B. Side Effects of Glucocorticoids In: Pharmacokinetics and Adverse Effects of Drugs, Dec. 20, 2017
ALI, M. CHAPTER 9—Pulmonary Drug Delivery. In: MALANGU, N. Handbook of Non-Invasive Drug Delivery Systems, 2010, p. 209-246
ALLEN, D. B. Inhaled Corticosteroids and Endocrine Effects in Childhood, Endocrinology and Metabolism Clinics of North America, v. 49, n. 4, p. 651-665, December 2020
ANEJA, R.; MUKERJEE, S. K.; SESHADRI, T. R. Synthesis of benzo-furan derivatives-I. Tetrahedron, 2, n. 3-4, p. 203-210, 1958.
AROOJ, M.; SAKKIAH, S.; CAO, G.; LEE, K. W. An innovative strategy for dual inhibitor design and its application in dual inhibition of human thymidylate synthase and dihydrofolate reductase enzymes. PLoS One, 8, no. 4, p. e60470, 2013.
Athanazio R. Airway disease: similarities and differences between asthma, COPD and bronchiectasis. Clinics (São Paulo). 2012; 67(11):1335-1343. doi:10.6061/clinics/2012(11)19.
AZAB, A.; NASSAR, A.; AZAB, A. N. Anti-Inflammatory Activity of Natural Products. Molecules, 21, n. 10, Oct. 1, 2016.
BABU, S.; NUTMAN, T. B. Proinflammatory cytokines dominate the early immune response to filarial parasites. J Immunol, 171, n. 12, p. 6723-6732, Dec. 15, 2003.
BANSAL, Y.; SETHI, P.; BANSAL, G. Coumarin: a potential nucleus for anti-inflammatory molecules. Medicinal Chemistry Research, 22, no. 7, p. 3049-3060, 2012.
BARNES, P. J. Th2 cytokines and asthma: an introduction. Respir Res, 2, n. 2, p. 64-65, 2001.
BARREIRO, E. J.; BOLZANI, V. d. S. Biodiversidade: fonte potencial para a descoberta de firmacos (Biodiversity: potential source for drug discovery). Quimica Nova, 32, n. 3, p. 679-688, 2009.
BARRIOS, R. J., et al., Asthma: Pathology and Pathophysiology. Arch Pathol Lab Med, 2006. 130(4): p. 447-51.
BARROS, T. A.; DE FREITAS, L. A.; FILHO, J. M.; NUNES, X. P. et al. Antinociceptive and anti-inflammatory properties of 7-hydroxycoumarin in experimental animal models: therapeutic potential for the control of inflammatory chronic pain. J Pharm Pharmacol, 62, n. 2, p. 205-213, February 2010.
BATEMAN, E. D.; O'BYRNE, P. M.; BUSSE, W. W.; LOTVALL, J. et al. Once-daily fluticasone furoate (FF)/vilanterol reduces risk of severe exacerbations in asthma versus FF alone. Thorax, 69, n. 4, p. 312-319, April 2014.
BAUMER, W.; HOPPMANN, J.; RUNDFELDT, C.; KIETZMANN, M. Highly selective phosphodiesterase 4 inhibitors for the treatment of allergic skin diseases and psoriasis. Inflamm Allergy Drug Targets, 6, n. 1, p. 17-26, March 2007.
BENDER, A. T.; BEAVO, J. A. Cyclic nucleotide phosphodiesterases: molecular regulation to clinical use. Pharmacol Rev, 58, n. 3, p. 488-520, September 2006.
Bolandi S M, Abdolmaleki Z, Assarehzadegan M A. Bevacizumab regulates inflammatory cytokines and inhibits VEGFR2 signaling pathway in an ovalbumin-induced rat model of airway hypersensitivity. Inflammopharmacology. 2021 June; 29(3):683-694. doi: 10.1007/s10787-021-00798-8. Epub 2021 Mar. 19. PMID: 33742375.
BOUSQUET, J.; BOUSQUET, P. J.; GODARD, P.; DAURES, J.-P. The public health implications of asthma. Bulletin of the World Health Organization, 83, 2005.
BOUSQUET, J.; CHANEZ, P.; LACOSTE, J. Y.; BARNEON, G. et al. Eosinophilic inflammation in asthma. N Engl J Med, 323, n. 15, p. 1033-1039, Oct. 11, 1990.
BOYTON, R. J.; ALTMANN, D. M. Asthma: new developments in cytokine regulation. Clin Exp Immunol, 136, n. 1, p. 13-14, April 2004.
BUIST A S. Similarities and differences between asthma and chronic obstructive pulmonary disease: treatment and early outcomes. Eur Respir J Suppl. 2003 January; 39:30s-35s. doi: 10.1183/09031936.03.00404903. PMID: 12572699.
CALHOUN, K. H. Asthma treatments: new and emerging therapies. Int Forum Allergy Rhinol, 5 Suppl 1, p. S76-81, September 2015.
CARDOSO, T. A.; RONCADA, C.; SILVA, E. R. D.; PINTO, L. A. et al. The impact of asthma in Brazil: a longitudinal analysis of data from a Brazilian national database system. J Bras Pneumol, 43, n. 3, p. 163-168, May-June 2017.
CAZZOLA, M.; MATERA, M. G. The effect of doxofylline in asthma and COPD. Respir Med, 164, p. 105904, April 2020.
CAZZOLA, M.; PAGE, C. P.; ROGLIANI, P.; MATERA, M. G. Beta2-agonist therapy in lung disease. Am J Respir Crit Care Med, 187, n. 7, p. 690-696, Apr. 1 2013.
CHARRIOT, J.; VACHIER, I.; HALIMI, L.; GAMEZ, A. S. et al. Future treatment for asthma. Eur Respir Rev, 25, n. 139, p. 77-92, March 2016.
CHEAH J. T. L. et al. The patient's perspective of the adverse effects of glucocorticoid use: A systematic review of quantitative and qualitative studies. From an OMERACT working group, Seminars in Arthritis and Rheumatism, v. 50, no. 5, p. 996-1005, October 2020
CHEENPRACHA, S.; PARK, E. J.; YOSHIDA, W. Y.; BARIT, C. et al. Potential anti-inflammatory phenolic glycosides from the medicinal plant Moringa oleifera fruits. Bioorg Med Chem, 18, n. 17, p. 6598-6602, Sep. 1 2010.
CHEN et al 2018. Inflammatory responses and inflammation-associated diseases in organs Oncotarget, 2018, 9(6), 7204-7218.
CHIANG, C. Y., et al., Osthole treatment improves Th2-mediated allergic asthma and exerts immunomodulatory effects on dendritic cell maturation and function. Cell Mol Immunol, 2017.
CORRY, D. B., et al., Requirements for allergen-induced airway hyperreactivity in T and B cell-deficient mice. Mol Med, 1998. 4(5): p. 344-55.
COSTA, R. S., et al., In vitro antileishmanial and antitrypanosomal activity of compounds isolated from the roots of Zanthoxylum tingoassuiba. Brazilian Journal of Pharmacognosy, 2018. 28(5): p. 551-558.
COUTINHO, A. E.; CHAPMAN, K. E. The anti-inflammatory and immunosuppressive effects of glucocorticoids, recent developments and mechanistic insights. Mol Cell Endocrinol, 335, n. 1, p. 2-13, Mar. 15, 2011.
CROMPTON, G., A brief history of inhaled asthma therapy over the last fifty years. Prim Care Respir J, 2006. 15(6): p. 326-31.
CURRAN, D. R. and L. Cohn, Advances in mucous cell metaplasia: a plug for mucus as a therapeutic focus in chronic airway disease. Am J Respir Cell Mol Biol, 2010. 42(3): p. 268-75.
DAHL, R. Systemic side effects of inhaled corticosteroids in patients with asthma. Respir Med, 100, n. 8, p. 1307-1317, August 2006.
DAUGHERTY, J.; LIN, X.; BAXTER, R.; SURUKI, R. et al. The impact of long-term systemic glucocorticoid use in severe asthma: A UK retrospective cohort analysis. J Asthma, 55, n. 6, p. 651-658, June 2018.
DE BOSSCHER, K. Mechanisms of anti-inflammatory action and of immunosuppression by glucocorticoids: negative interference of activated glucocorticoid receptor with transcription factors. Journal of Neuroimmunology, 109, no. 1, p. 16-22, 2000.
DE LIMA, F. O.; NONATO, F. R.; COUTO, R. D.; BARBOSA FILHO, J. M. et al. Mechanisms involved in the antinociceptive effects of 7-hydroxycoumarin. J Nat Prod, 74, n. 4, p. 596-602, Apr. 25 2011.
DESAI, J. T.; DESAI, C. K.; DESAI, K. R. A convenient, rapid and eco-friendly synthesis of isoxazoline heterocyclic moiety containing bridge at 2°-amine as potential pharmacological agent. Journal of the Iranian Chemical Society, 5, no. 1, p. 67-73, 2008.
DINARELLO, C. A. Overview of the IL-1 family in innate inflammation and acquired immunity. Immunol Rev, 281, n. 1, p. 8-27, January 2018.
IV Diretrizes Brasileiras para o Manejo da Asma (IV Brazilian Guidelines for Asthma Management). J Bras Pneumol. 2006; 32(Suppl 7):S 447-S 474
DONAHUE, J. G.; WEISS, S. T.; LIVINGSTON, J. M.; GOETSCH, M. A. et al. Inhaled steroids and the risk of hospitalization for asthma. JAMA, 277, n. 11, p. 887-891, Mar. 19, 1997.
DRAY, A.; URBAN, L.; DICKENSON, A. Pharmacology of chronic pain. Trends in Pharmacological Sciences, 15, n. 6, p. 190-197, 1994.
DUMONDE, D. C.; WOLSTENCROFT, R. A.; PANAYI, G. S.; MATTHEW, M. et al. “Lymphokines”: non-antibody mediators of cellular immunity generated by lymphocyte activation. Nature, 224, n. 5214, p. 38-42, Oct. 4, 1969.
FIREMAN, P. B2 Agonists and Their Safety in the Treatment of Asthma. Allergy Proceedings, 16, no. 5, 1995.
FISCHER, P. M. Computational chemistry approaches to drug discovery in signal transduction. Biotechnol J, 3, n. 4, p. 452-470, April 2008.
FRANCO, R.; NASCIMENTO, H. F.; CRUZ, A. A.; SANTOS, A. C. et al. The economic impact of severe asthma on low-income families. Allergy, 64, n. 3, p. 478-483, March 2009.
FYLAKTAKIDOU, K. C.; HADJIPAVLOU-LITINA, D. J.; LITINAS, K. E.; NICOLAIDES, D. N. Natural and synthetic coumarin derivatives with anti-inflammatory/antioxidant activities. Curr Pharm Des, 10, n. 30, p. 3813-3833, 2004.
GIBSON, P. G.; POWELL, H.; WILSON, A.; ABRAMSON, M. J. et al. Self-management education and regular practitioner review for adults with asthma. Cochrane Database of Systematic Reviews, 2002.
GOMEZ-OUTES, A.; SUAREZ-GEA, M. L.; CALVO-ROJAS, G.; LECUMBERRI, R. et al. Discovery of anticoagulant drugs: a historical perspective. Curr Drug Discov Technol, 9, n. 2, p. 83-104, Jun. 1 2012.
GUILBERT, T. W.; MORGAN, W. J.; ZEIGER, R. S.; MAUGER, D. T. et al. Long-term inhaled corticosteroids in preschool children at high risk for asthma. N Engl J Med, 354, n. 19, p. 1985-1997, May 11, 2006.
HADDAD, J. J. On the enigma of pain and hyperalgesia: A molecular perspective. Biochem Biophys Res Commun, 353, no. 2, p. 217-224, Feb. 9, 2007.
HALDAR, P.; BRIGHTLING, C. E.; SINGAPURI, A.; HARGADON, B. et al. Outcomes after cessation of mepolizumab therapy in severe eosinophilic asthma: a 12-month follow-up analysis. J Allergy Clin Immunol, 133, n. 3, p. 921-923, March 2014.
HARVEY, A. L.; EDRADA-EBEL, R.; QUINN, R. J. The re-emergence of natural products for drug discovery in the genomics era. Nat Rev Drug Discov, 14, n. 2, p. 111-129, February 2015.
HEFFLER, E. et al. Inhaled Corticosteroids Safety and Adverse Effects in Patients with Asthma, The Journal of Allergy and Clinical Immunology: In Practice, vol. 6, no. 3, p. 776-781, Feb. 2, 2018.
EIXARCH, H.; HALTNER-UKOMADU, H.; BEISSWENGER, C.; BOCK, U. Drug Delivery to the Lung: Permeability and Physicochemical Characteristics of Drugs as the Basis for a Pulmonary Biopharmaceutical Classification System (pBCS), J Epithel Biol Pharmacol, v. 3, p. 1-14, 2010.
Hisbello S. Campos. Asthma: its origins, inflammatory mechanisms and the role of the corticosteroid. Rev Bras Pneumol Sanit 2007; 15(1):47-60.
HOUSLAY, M. D.; SCHAFER, P.; ZHANG, K. Y. J. Keynote review: Phosphodiesterase-4 as a therapeutic target. Drug Discovery Today, 10, no. 22, p. 1503-1519, 2005.
HUSCHER, D.; THIELE, K.; GROMNICA-IHLE, E.; HEIN, G. et al. Dose-related patterns of glucocorticoid-induced side effects. Ann Rheum Dis, 68, n. 7, p. 1119-1124, July 2009.
IZUHARA, K.; Ohta, S.; SHIRAISHI, H.; SUZUKI, S. et al. The mechanism of mucus production in bronchial asthma. Curr Med Chem, 16, n. 22, p. 2867-2875, 2009.
JANKOWSKA, A.; SWIERCZEK, A.; CHLON-RZEPA, G.; PAWLOWSKI, M. et al. PDE7-Selective and Dual Inhibitors: Advances in Chemical and Biological Research. Curr Med Chem, 24, n. 7, p. 673-700, 2017.
Jeffery P K. Comparison of the structural and inflammatory characteristics of COPD and asthma. Giles F. Filley Lecture. Chest. 2000 May; 117(5 Suppl 1):251S-60S. doi: 10.1378/chest.117.5_suppl_1.251s. PMID: 10843939.
JI, H. F.; LI, X. J.; ZHANG, H. Y. Natural products and drug discovery. Can thousands of years of ancient medical knowledge lead us to new and powerful drug combinations in the fight against cancer and dementia?EMBO Rep, 10, n. 3, p. 194-200, March 2009.
KELLER, A. C.; RODRIGUEZ, D.; RUSSO, M. Nitric oxide paradox in asthma. Mem Inst Oswaldo Cruz, 100 Suppl 1, p. 19-23, March 2005.
KELLY, H.; NELSON, H. Potential adverse effects of the inhaled corticosteroids. Journal of Allergy and Clinical Immunology, 112, no. 3, p. 469-478, 2003.
KELSO, A. Cytokines: principles and prospects. Immunol Cell Biol, 76, no. 4, p. 300-317, August 1998.
KIM, H. P.; SON, K. H.; CHANG, H. W.; KANG, S. S. Anti-inflammatory plant flavonoids and cellular action mechanisms. J Pharmacol Sci, 96, n. 3, p. 229-245, November 2004.
KITCHEN, D. B.; DECORNEZ, H.; FURR, J. R.; BAJORATH, J. Docking and scoring in virtual screening for drug discovery: methods and applications. Nat Rev Drug Discov, 3, n. 11, p. 935-949, November 2004.
KONTOGIORGIS, C. A.; HADJIPAVLOU-LITINA, D. J. Synthesis and antiinflammatory activity of coumarin derivatives. J Med Chem, 48, n. 20, p. 6400-6408, Oct. 6, 2005.
KUMAR, R. K., et cal., Inhibition of inflammation and remodeling by roflumilast and dexamethasone in murine chronic asthma. J Pharmacol Exp Ther, 2003. 307(1): p. 349-55.
KUPELI AKKOL, E.; GENC, Y.; KARPUZ, B.; SOBARZO-SANCHEZ, E. et al. Coumarins and Coumarin-Related Compounds in Pharmacotherapy of Cancer. Cancers (Basel), 12, no. 7, Jul. 19, 2020.
Li A, Chan H P, Gan P X L, Liew M F, Wong W S F, Lim H F. Eosinophilic endotype of chronic obstructive pulmonary disease: similarities and differences from asthma. Korean J Intern Med. 2021 November; 36(6):1305-1319. doi: 10.3904/kjim.2021.180. Epub 2021 October 12. PMID: 34634855; PMCID: PMC8588979.
LI, D. and L. WU, Coumarins from the roots of Angelica dahurica cause anti-allergic inflammation. Exp Ther Med, 2017. 14(1): p. 874-880
LIN, E.; CALVANO, S. E.; LOWRY, S. F. Inflammatory cytokines and cellular response in surgery. Surgery, 127, n. 2, p. 117-126, February 2000.
LIN, T. T.; HUANG, Y. Y.; TANG, G. H.; CHENG, Z. B. et al. Prenylated coumarins: natural phosphodiesterase-4 inhibitors from Toddalia asiatica. J Nat Prod, 77, n. 4, p. 955-962, Apr. 25 2014.
LIPWORTH, B. J., Pharmacokinetics of inhaled drugs. Br J Clin Pharmacol, 1996. 42(6): p. 697-705.
LIPWORTH, B. J. Phosphodiesterase-4 inhibitors for asthma and chronic obstructive pulmonary disease. The Lancet, 365, no. 9454, p. 167-175, 2005.
MADORE, A. M.; LAPRISE, C. Immunological and genetic aspects of asthma and allergy. J Asthma Allergy, 3, p. 107-121, Aug. 20 2010.
MALIKOV, V. M.; SAIDKHODZHAEV, A. I.; ARIPOV, K. N. Coumarins: Plants, structure, properties. Chemistry of Natural Compounds, 34, no. 2, p. 202-264, 1998.
Mattos M S, Ferrero M R, Kraemer L, Lopes G A O, Reis D C, Cassali G D, Oliveira F M S, Brandolini L, Allegretti M, Garcia C C, Martins M A, Teixeira M M, Russo R C. CXCR1 and CXCR2 Inhibition by Ladarixin Improves Neutrophil-Dependent Airway Inflammation in Mice. Front Immunol. 2020 Oct. 2; 11:566953. doi: 10.3389/fimmu.2020.566953. PMID: 33123138; PMCID: PMC7566412.
MCBRIEN, C. N.; MENZIES-GOW, A. The Biology of Eosinophils and Their Role in Asthma. Front Med (Lausanne), 4, p. 93, 2017.
MEDZHITOV, R. Inflammation 2010: new adventures of an old flame. Cell, 140, no. 6, p. 771-776, Mar. 19 2010.
MENG, X. Y.; ZHANG, H. X.; MEZEI, M.; CUI, M. Molecular Docking: A powerful approach for structure-based drug discovery. Curr Comput Aided Drug Des, 7, no. 2, p. 146-157, June 2011.
MENZIES-GOW, A.; FLOOD-PAGE, P.; SEHMI, R.; BURMAN, J. et al. Anti-IL-5 (mepolizumab) therapy induces bone marrow eosinophil maturational arrest and decreases eosinophil progenitors in the bronchial mucosa of atopic asthmatics. J Allergy Clin Immunol, 111, n. 4, p. 714-719, April 2003.
MIGUEL, M. G. Antioxidant and anti-inflammatory activities of essential oils: a short review. Molecules, 15, n. 12, p. 9252-9287, Dec. 15, 2010.
MIRAVITLLES, M. et al. Systematic review on long-term adverse effects of inhaled corticosteroids in the treatment of COPD, European Respiratory Review, 30: 210075, 2021
MORE, D. H.; MAHULIKAR, P. P. Microwave assisted one-pot synthesis of nitrogen and oxygen containing heterocycles from acyl meldrum's acid. Indian Journal of Chemistry, 50, 2011.
NEWMAN, D. J.; CRAGG, G. M. Natural Products as Sources of New Drugs over the Nearly Four Decades from 01/1981 to 09/2019. J Nat Prod, 83, n. 3, p. 770-803, Mar. 27 2020.
OGUNGBE, I. V.; ERWIN, W. R.; SETZER, W. N. Antileishmanial phytochemical phenolics: molecular docking to potential protein targets. J Mol Graph Model, 48, p. 105-117, March 2014.
OKIN, D.; MEDZHITOV, R. Evolution of inflammatory diseases. Curr Biol, 22, n. 17, p. R733-740, Sep. 11, 2012.
ORTEGA, H. G.; LIU, M. C.; PAVORD, I. D.; BRUSSELLE, G. G. et al. Mepolizumab treatment in patients with severe eosinophilic asthma. N Engl J Med, 371, n. 13, p. 1198-1207, Sep. 25, 2014.
PAGE, C. P. An explanation of the asthma paradox. Am Rev Respir Dis, 147, n. 6 Pt 2, p. S29-32, June 1993.
PATRIDGE, E.; GAREISS, P.; KINCH, M. S.; HOYER, D. An analysis of FDA-approved drugs: natural products and their derivatives. Drug Discov Today, 21, n. 2, p. 204-207, February 2016.
PATYAR, S.; PRAKASH, A.; MEDHI, B. Dual inhibition: a novel promising pharmacological approach for different disease conditions. J Pharm Pharmacol, 63, n. 4, p. 459-471, April 2011.
PAWANKAR, R. Mast cell function modulating IgE-mediated allergy. Allergology International, 48, no. 3, p. 171-182, 1999.
POSSA, S. S., et al., Eosinophilic inflammation in allergic asthma. Front Pharmacol, 2013. 4: p. 46.
PUNCHARD, N. A.; WHELAN, C. J.; ADCOCK, I. The Journal of Inflammation. J Inflamm (Lond), 1, n. 1, p. 1, Sep. 27, 2004.
R. Hays, S. and J. V. Fahy, The role of mucus in fatal asthma. The American Journal of Medicine, 2003. 115(1): p. 68-69.
RAKOTOARISON, O.; RABENAU, I.; LOBSTEIN, A.; UM, B. H. et al. Vasorelaxing properties and bio-guided fractionation of Cedrelopsis strikei. Planta Med, 69, n. 2, p. 179-181, February 2003.
RATHER, L. J. Disturbance of function (functio laesa): the legendary fifth cardinal sign of inflammation, added by Galen to the four cardinal signs of Celsus. Bull N Y Acad Med, 47, n. 3, p. 303-322, March 1971.
ROIFMAN, I.; BECK, P. L.; ANDERSON, T. J.; EISENBERG, M. J. et al. Chronic inflammatory diseases and cardiovascular risk: a systematic review. Can J Cardiol, 27, n. 2, p. 174-182, March-April 2011.
RUGE, A. C.; KIRCH, J.; LEHR, C. M. Pulmonary drug delivery: from generating aerosols to overcoming biological barriers therapeutic possibilities and technological challenges, The Lancet Respiratory Medicine, v. 1, i. 5, p. 402-413, Jul. 1, 2013
SALARI, P.; ABDOLLAHI, M. Phosphodiesterase inhibitors in inflammatory bowel disease. Expert Opin Investig Drugs, 21, n. 3, p. 261-264, March 2012.
SANTOS, W. A.; DOURADO, K. M. C.; ARAUJO, F. A.; JESUS, R. L. C. et al. Braylin induces a potent vasorelaxation, involving distinct mechanisms in superior mesenteric and iliac arteries of rats. Naunyn Schmiedebergs Arch Pharmacol, Oct. 9, 2020.
SCHOFIELD, M. L. Asthma pharmacotherapy. Otolaryngol Clin North Am, 47, n. 1, p. 55-64, February 2014.
SCIO, E.; RIBEIRO, A.; ALVES, T. M.; ROMANHA, A. J. et al. New bioactive coumarins from Kielmeyera albopunctata. J Nat Prod, 66, n. 5, p. 634-637, May 2003.
SEREZANI, C. H.; BALLINGER, M. N.; ARONOFF, D. M.; PETERS-GOLDEN, M. Cyclic AMP: master regulator of innate immune cell function. Am J Respir Cell Mol Biol, 39, n. 2, p. 127-132, August 2008.
SHUBAYEV, V. I.; KATO, K.; MYERS, R. R. Translational Pain Research: From Mouse to Man. Boca Raton (FL): CRC Press/Taylor & Francis, 2010.
SILVEIRA, M. R.; NUNES, K. P.; CARA, D. C.; SOUZA, D. G. et al. Infection with Strongyloides venezuelensis induces transient airway eosinophilic inflammation, an increase in immunoglobulin E, and hyperresponsiveness in rats. Infect Immun, 70, n. 11, p. 6263-6272, November 2002.
SOUTHAM, D. S., et al., Goblet cell rebound and airway dysfunction with corticosteroid withdrawal in a mouse model of asthma. Am J Respir Crit Care Med, 2008. 178(11): p. 1115-22.
STRAND, V.; FIORENTINO, D.; HU, C.; DAY, R. M. et al. Improvements in patient-reported outcomes with apremilast, an oral phosphodiesterase 4 inhibitor, in the treatment of moderate to severe psoriasis: results from a phase IIb randomized, controlled study. Health Qual Life Outcomes, 11, p. 82, May 10 2013.
SUISSA, S.; ERNST, P.; BENAYOUN, S.; BALTZAN, M. et al. Low-dose inhaled corticosteroids and the prevention of death from asthma. N Engl J Med, 343, n. 5, p. 332-336, Aug. 3 2000.
SUN, C.; ZHAO, W.; WANG, X.; SUN, Y. et al. A pharmacological review of dicoumarol: An old natural anticoagulant agent. Pharmacol Res, 160, p. 105193, October 2020.
TRACY, R. P. The five cardinal signs of inflammation: Heat, Pain, Flushing, Tumor and Penuria (Apologies to Aulus Cornelius Celsus, De Medicina, c. A.D. 25). J Gerontol A Biol Sci Med Sci, 61, n. 10, p. 1051-1052, October 2006.
TSAI, I.-L.; WUN, M.-F.; TENG, C.-M.; ISHIKAWA, T. et al. Anti-platelet aggregation constituents from formosan Toddalia asiatica. Phytochemistry, 48, n. 8, p. 1377-1382, 1998.
VASCONCELOS, J. F., et al., Effects of umbelliferone in a murine model of allergic airway inflammation. Eur J Pharmacol, 2009. 609(1-3): p. 126-31.
VERRI, W. A., Jr.; CUNHA, T. M.; PARADA, C. A.; POOLE, S. et al. Hypernociceptive role of cytokines and chemokines: targets for analgesic drug development?Pharmacol Ther, 112, n. 1, p. 116-138, October 2006.
WANG, J., et al., Anti-asthmatic activity of osthole in an ovalbumin-induced asthma murine model. Respir Physiol Neurobiol, 2017. 239: p. 64-69.
WATZL, B. Anti-inflammatory effects of plant-based foods and of their constituents. Int J Vitam Nutr Res, 78, n. 6, p. 293-298, December 2008.
WIDDICOMBE JG (2003) Overview of neural pathways in allergy and asthma. Pulm Pharmacol Ther 16(23):30. https://doi. org/10. 1016/s1094-5539(02) 00178-5
WITAICENIS, A.; SEITO, L. N.; DA SILVEIRA CHAGAS, A.; DE ALMEIDA, L. D., Jr. et al. Antioxidant and intestinal anti-inflammatory effects of plant-derived coumarin derivatives. Phytomedicine, 21, n. 3, p. 240-246, Feb. 15 2014.
XIONG, Y., et al., The effects of nodakenin on airway inflammation, hyper-responsiveness and remodeling in a murine model of allergic asthma. Immunophannacol Immunotoxicol, 2014. 36(5): p. 341-8.
YOO, S. W., et al., Constituents of the fruits and leaves of Euodia daniellii. Arch Pharm Res, 2002. 25(6): p. 824-30.
ZHANG, J. M.; AN, J. Cytokines, inflammation, and pain. Int Anesthesiol Clin, 45, n. 2, p. 27-37, Spring 2007.

1. Use of an active compound of Formula I: 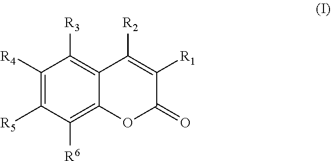
wherein any one of R₁, R₂, R₃, R₄, R₅, R₆, is selected from the group consisting of H, OH, O, S, N, C₁-C₅ alkyl, C₂-C₅ alkenyl, C₂-C₅ alkynyl, optionally aromatic C₃-C₇ cycloalkyl, C₁-C₅ heteroalkyl and optionally aromatic C₃-C₇ heterocycloalkyl; wherein any of C₁-C₅ alkyl, C₂-C₅ alkenyl, C₂-C₅ alkynyl, optionally aromatic C₃-C₇ cycloalkyl, C₁-C₅ heteroalkyl and optionally aromatic C₃-C₇ heterocycloalkyl may be optionally substituted with one or more substituents selected from OH, O, S, N, C₁-C₅ alkyl, C₂-C₅ alkenyl, C₂-C₅ alkynyl, optionally aromatic C₃-C₇ cycloalkyl, C₁-C₅ heteroalkyl and optionally aromatic C₃-C₇ heterocycloalkyl; and in which C₁-C₅ heteroalkyl and optionally aromatic C₃-C₇ heterocycloalkyl contain in the chain thereof 1 to 3 heteroatoms selected from F, O, N, Cl, Br, I, S; and/or
R₃ and R₄, R₄ and R₅, R₅ and R₆ are independently taken together to form an optionally aromatic 3 to 7 membered cyclic group which may contain 1 to 3 heteroatoms selected from O, N, S as ring members, the cyclic group being optionally substituted with one or more substituents selected from OH, O, S, N, C₁-C₅ alkyl, C₂-C₅ alkenyl, C₂-C₅ alkynyl, optionally aromatic C₃-C₇ cycloalkyl, C₁-C₅ heteroalkyl and optionally aromatic C₃-C₇ heterocycloalkyl in which C₁-C₅ heteroalkyl and optionally aromatic C₃-C₇ heterocycloalkyl contain in their chain 1 to 3 heteroatoms selected from F, O, N, Cl, Br, I, S;
or salts, prodrugs, stereoisomers, hydrates, dimeric derivatives, isosteres, bioisosteres, and polymorphic forms thereof, characterized in that it is for the manufacture of a medicine for the treatment of asthma or Chronic Obstructive Pulmonary Disease (COPD).

2. Use, according to claim 1, characterized in that the active compound is selected from the compound of Formula II: 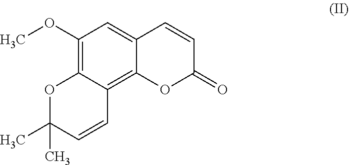 [SMILES: COc1cc2ccc(=O)oc2c2c1OC(C)(C)C=C2]
or salts, prodrugs, stereoisomers, hydrates, dimeric derivatives, isosteres, bioisosteres, and polymorphic forms thereof.

3. Use, according to claim 1, characterized in that the active compound is braylin or its pharmaceutically acceptable salts.

4. Use, according to claim 1, characterized in that the medicine is formulated in a form suitable for administration via inhalation.

5. Use, according to claim 1, characterized in that the medicine contains from 1 to 1,000 mg of the active compound.

6. Use, according to claim 1, characterized in that the medicine is in the form of a powder, fine granules, solution or suspension.

7. Use, according to claim 6, characterized in that the medicine is formulated in a form suitable for administration by capsule, spray or aerosol.

8.-15. (canceled)

16. Method of treating asthma and/or COPD, characterized in that it comprises administering a therapeutically effective amount of a compound of Formula I: 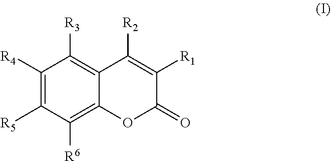
wherein any one of R₁, R₂, R₃, R₄, R₅, R₆, is selected from the group consisting of H, OH, O, S, N, C₁-C₅ alkyl, C₂-C₅ alkenyl, C₂-C₅ alkynyl, optionally aromatic C₃-C₇ cycloalkyl, C₁-C₅ heteroalkyl and C₃-C₇ heterocycloalkyl optionally aromatic; wherein any of C₁-C₅ alkyl, C₂-C₅ alkenyl, C₂-C₅ alkynyl, optionally aromatic C₃-C₇ cycloalkyl, C₁-C₅ heteroalkyl and optionally aromatic C₃-C₇ heterocycloalkyl may be optionally substituted with one or more substituents selected from OH, O, S, N, C₁-C₅ alkyl, C₂-C₅ alkenyl, C₂-C₅ alkynyl, optionally aromatic C₃-C₇ cycloalkyl, C₁-C₅ heteroalkyl and optionally aromatic C₃-C₇ heterocycloalkyl; and in which C₁-C₅ heteroalkyl and optionally aromatic C₃-C₇ heterocycloalkyl contain in their chain 1 to 3 heteroatoms selected from F, O, N, Cl, Br, I, S; and/or
R₃ and R₄, R₄ and R₅, R₅ and R₆ are independently taken together to form an optionally aromatic 3 to 7 membered cyclic group which may contain 1 to 3 heteroatoms selected from O, N, S as ring members, the cyclic group being optionally substituted with one or more substituents selected from OH, O, S, N, C₁-C₅ alkyl, C₂-C₅ alkenyl, C₂-C₅ alkynyl, optionally aromatic C₃-C₇ cycloalkyl, C₁-C₅ heteroalkyl and optionally aromatic C₃-C₇ heterocycloalkyl in which C₁-C₅ heteroalkyl and optionally aromatic C₃-C₇ heterocycloalkyl contain in the chain thereof 1 to 3 heteroatoms selected from F, O, N, Cl, Br, I, S;
or salts, prodrugs, stereoisomers, hydrates, dimeric derivatives, isosteres, bioisosteres, and polymorphic forms thereof to a patient in need of the same.

17. Method according to claim 16, characterized in that the compound is selected from the compound of Formula II: 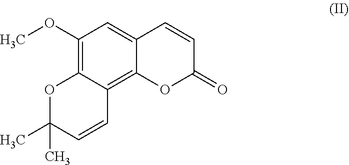 [SMILES: COc1cc2ccc(=O)oc2c2c1OC(C)(C)C=C2]
or salts, prodrugs, stereoisomers, hydrates, dimeric derivatives, isosteres, bioisosteres, and polymorphic forms thereof.

18. Method according to claim 16, characterized in that the compound is braylin or its pharmaceutically acceptable salts.

19. Method according to claim 16, characterized in that the compound is administered at a dose of 1 to 100 mg/kg.

20. Method according to claim 16, characterized in that the compound is administered at a dose of 50 mg/kg.

21. Method according to claim 16, characterized in that the compound is administered via inhalation.

22. Method according to claim 16, characterized in that the compound is administered in the form of a powder, fine granules, solution or suspension.

23. Method according to claim 16, characterized in that the compound is administered by capsule, spray or aerosol.

24. Compound characterized in that it is of Formula I: 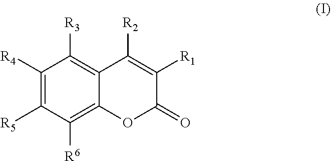
wherein any one of R₁, R₂, R₃, R₄, R₅, R₆, is selected from the group consisting of H, OH, O, S, N, C₁-C₅ alkyl, C₂-C₅ alkenyl, C₂-C₅ alkynyl, cycloalkyl C₃-C₇ optionally aromatic, C₁-C₅ heteroalkyl and C₃-C₇ heterocycloalkyl optionally aromatic; wherein any of C₁-C₅ alkyl, C₂-C₅ alkenyl, C₂-C₅ alkynyl, optionally aromatic C₃-C₇ cycloalkyl, C₁-C₅ heteroalkyl and optionally aromatic C₃-C₇ heterocycloalkyl may be optionally substituted with one or more substituents selected from OH, O, S, N, C₁-C₅ alkyl, C₂-C₅ alkenyl, C₂-C₅ alkynyl, optionally aromatic C₃-C₇ cycloalkyl, C₁-C₅ heteroalkyl and optionally aromatic C₃-C₇ heterocycloalkyl; and in which C₁-C₅ heteroalkyl and optionally aromatic C₃-C₇ heterocycloalkyl contain in the chain thereof 1 to 3 heteroatoms selected from F, O, N, Cl, Br, I, S; and/or
R₃ and R₄, R₄ and R₅, R₅ and R₆ are independently taken together to form an optionally aromatic 3 to 7 membered cyclic group which may contain 1 to 3 heteroatoms selected from O, N, S as ring members, the cyclic group being optionally substituted with one or more substituents selected from OH, O, S, N, C₁-C₅ alkyl, C₂-C₅ alkenyl, C₂-C₅ alkynyl, optionally aromatic C₃-C₇ cycloalkyl, C₁-C₅ heteroalkyl and optionally aromatic C₃-C₇ heterocycloalkyl in which C₁-C₅ heteroalkyl and optionally aromatic C₃-C₇ heterocycloalkyl contain in the chain thereof 1 to 3 heteroatoms selected from F, O, N, Cl, Br, I, S;
or salts, prodrugs, stereoisomers, hydrates, dimeric derivatives, isosteres, bioisosteres, and polymorphic forms thereof,
for use in the treatment of asthma and/or COPD.

25. Compound, according to claim 24, characterized in that it is of Formula II: 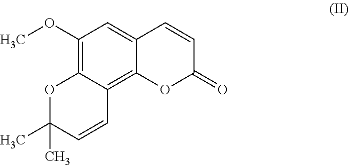 [SMILES: COc1cc2ccc(=O)oc2c2c1OC(C)(C)C=C2]
or salts, prodrugs, stereoisomers, hydrates, dimeric derivatives, isosteres, bioisosteres, and polymorphic forms thereof.

26. Compound according to claim 24, characterized in that the compound is braylin or the pharmaceutically acceptable salts thereof.

27. Pharmaceutical formulation characterized in that it comprises the active compound of claim 24, and at least one pharmaceutically acceptable additive.

28. Pharmaceutical formulation according to claim 27, characterized in that the active compound is selected from the compound of Formula II: 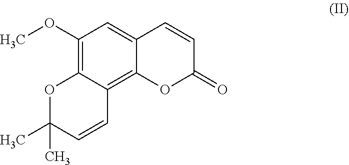 [SMILES: COc1cc2ccc(=O)oc2c2c1OC(C)(C)C=C2]
or salts, prodrugs, stereoisomers, hydrates, dimeric derivatives, isosteres, bioisosteres, and polymorphic forms thereof.
